(19) United States
(12) Patent Application Publication
(10) Pub. No.: US 2021/0316086 A1
(43) Pub. Date: Oct. 14, 2021

(54) PORTABLE MEDICAL SPRAY

(71) Applicant: Chung-Sing LIN, Tainan (TW)

(72) Inventor: Chung-Sing LIN, Tainan (TW)

(73) Assignee: Chung-Sing LIN, Tainan (TW)

(21) Appl. No.: 16/843,895

(22) Filed: Apr. 9, 2020

(51) Int. Cl. A61M 11/00; A61M 39/22

(52) U.S. Cl. CPC A61M 11/007; A61M 2210/0618; A61M 39/22

(57) ABSTRACT

A portable medical spray includes a tubular body, and a canister is located in the room. A needle is located in the room and a nozzle is formed to the top end of the body. The nozzle has a passage defined therethrough and the passage communicates with the room and the needle. A holding portion is formed to outside of the body. A connection portion extends from the bottom end of the body. The canister has medicine received therein and a breakable portion is formed to the top end of the canister and located corresponding to the needle. An end part is movable movably mounted to the connection portion. When a user pushes the end part to move the canister toward the needle, the needle penetrates through the breakable portion of the canister to release the medicine via the needle.

### BACKGROUND OF THE INVENTION

### 1. Fields of the invention

The present invention relates to a medical spray, and more particularly, to a portable medical spray.

### 2. Descriptions of Related Art

The conventional medical spray is used to soothe uncomfortable symptoms caused by rhinitis, stuffy nose, throat ache and the like, and the medical spray is inserted into the patient's nose or mouth to spray medicine onto the infected areas. When spraying the medicine, a compressor and a spray are needed to send medicine to the desired areas. The equipment may include multiple parts and occupy a lot of space. In addition, theses equipment may not be conveniently obtained in remote areas.

The present invention intends to provide a portable medicine spray to eliminate the shortcomings mentioned above.

### SUMMARY OF THE INVENTION

The present invention relates to a portable medical spray and comprises a body having a room defined therein. A needle is located in the room. A nozzle is formed to the top end of the body and has a passage defined through the nozzle. The passage communicates with the room and is located corresponding to the needle. A holding portion is formed to outside of the body. A connection portion extends from the bottom end of the body. A top groove and a bottom groove are respectively formed to outside of the connection portion.

A canister is located in the room and has medicine received therein. A breakable portion is formed to the top end of the canister and located corresponding to the needle.

An end part has a reception space defined in the top end thereof. A top ridge and a bottom ridge are respectively formed to the inner periphery of the reception space. The end part is movably mounted to the connection portion. When pushing the end part to move the canister toward the needle, the top and bottom ridges of the end cap are engaged with the bottom groove and the top groove in sequence.

Preferably, the passage of the nozzle of the body has a valve received therein. An adjustment knob is connected to the nozzle and operates the valve.

Preferably, the canister is made by metal, and the breakable portion of the canister is made of rubber.

The primary object of the present invention is to provide a portable medical spray which is compact for easy carry. The canister can be replaced to include different medicine therein.

The present invention will become more obvious from the following description when taken in connection with the accompanying drawings which show, for purposes of illustration only, a preferred embodiment in accordance with the present invention.

### BRIEF DESCRIPTION OF THE DRAWINGS

FIG. 1 is an exploded view to show the portable medical spray of the present invention;

FIG. 2 is a perspective view to show the portable medical spray of the present invention, wherein the canister is not yet pushed toward the needle;

FIG. 3 is a cross sectional view of the portable medical spray of the present invention;

FIG. 4 is a perspective view to show that the portable medical spray of the present invention is used as a nasal spray;

FIG. 5 is a cross sectional view to show that the canister is pushed toward the needle by pushing the end part toward the needle, and the needle penetrates through the breakable portion to eject the medicine in the canister from the nozzle of the portable medical spray of the present invention, and

FIG. 6 is a perspective view of the portable medical spray of the present invention, wherein the canister is pushed toward the needle.

### DETAILED DESCRIPTION OF THE INVENTION

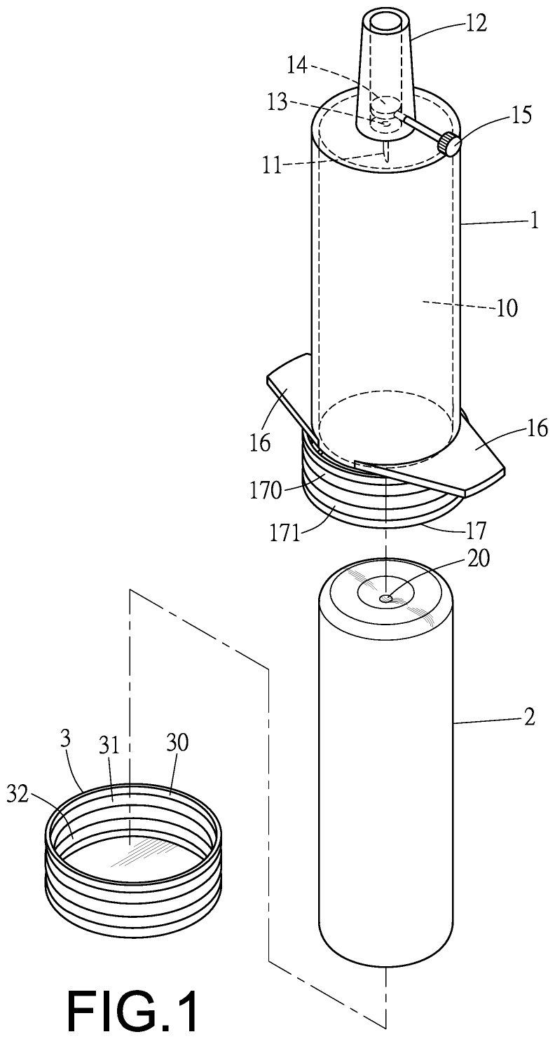

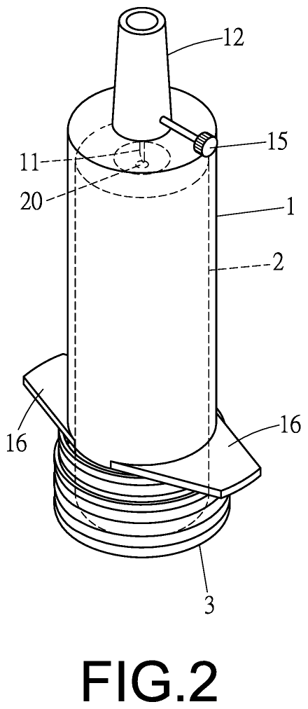

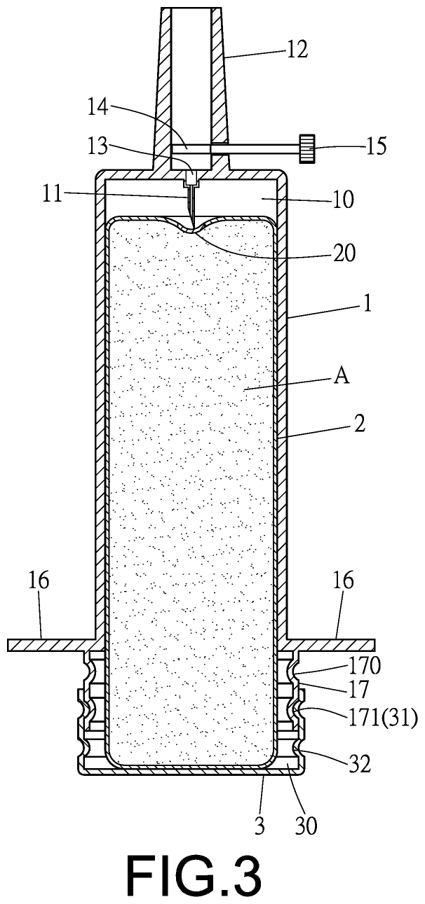

Referring to FIGS. 1 to 3, the portable medical spray of the present invention comprises a tubular body 1 having a room 10 defined therein. A needle 11 is located in the room 10 and a nozzle 12 is formed to the top end of the body 1. A passage 13 is defined through the nozzle 12 and 13 communicates with the room 10 and is located corresponding to the needle 11. A holding portion 16 is formed to outside of the body 1 and includes two finger flanges. A connection portion 17 extends from the bottom end of the body 1. A top groove 170 and a bottom groove 171 are respectively formed to outside of the connection portion 17.

A canister 2 is located in the room 10 and has medicine “A” received therein. The medicine “A” is stored in the canister 2 under pressure, and the amount of the medicine “A” is for one-time use only. A breakable portion 20 is formed to the top end of the canister 2 and located corresponding to the needle 11. Preferably the canister 2 is made by metal, and the breakable portion 20 is made by rubber. The breakable portion 20 of the canister 2 is made by rubber. A valve 14 is located in the nozzle 12 and an adjustment knob 15 is connected with a rod which extends through the nozzle 12 and is connected to the valve 14 so that when the user rotates the adjustment knob 15, the valve 14 is operated and adjusted. An end part 3 has a reception space 130 defined in the top end thereof, and the end part 3 has a closed bottom end. A top ridge 31 and a bottom ridge 32 are respectively formed to the inner periphery of the reception space 130. The end part 3 is movably mounted to the connection portion 17. The top ridge 31 of the end part 3 is engaged with the bottom groove 171 of the connection portion 17 when the end part 3 is not yet pushed by the user.

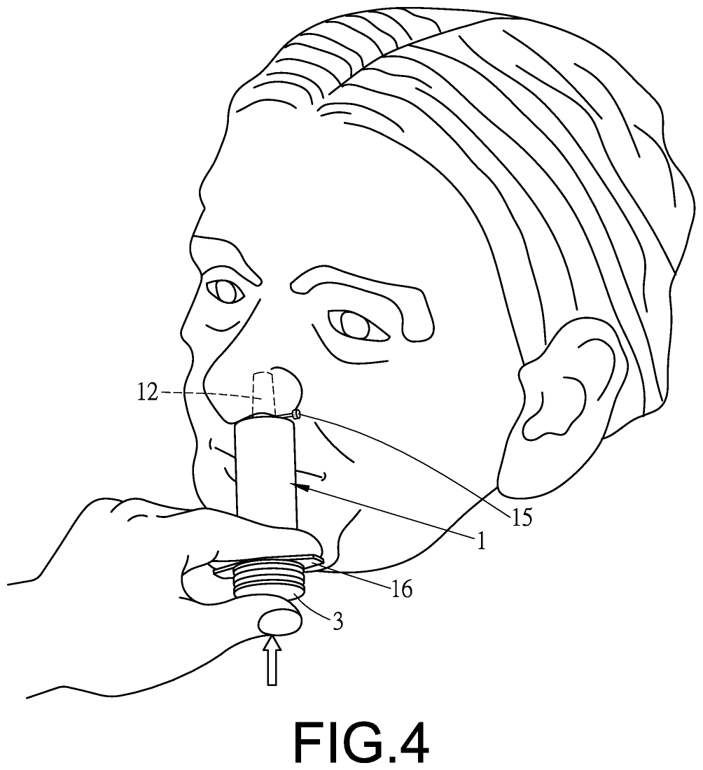

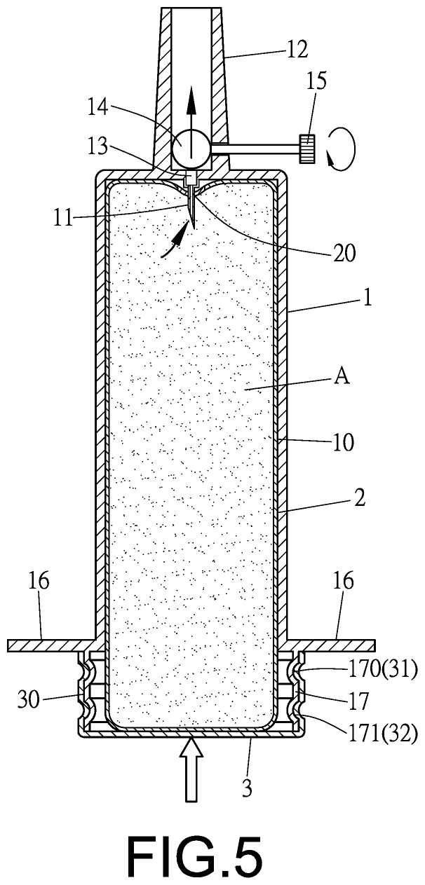

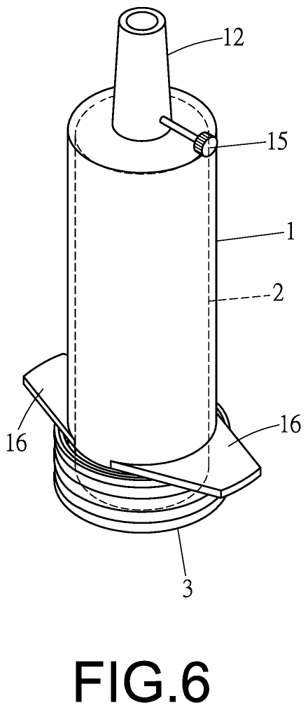

As shown in FIGS. 4 to 6, when in use, the user uses two fingers to holds the body 1 and the two fingers put on the finger flanges of the holding portion 16. The nozzle 12 is then inserted into the patient's nose as shown in FIG. 4. The user then uses the thumb to push the end part 3 toward the needle 11 or the nozzle 12. As shown in FIG. 5, the top ridge 31 of the end part 3 is then moved and engaged with the top groove 170 of the connection portion 17, and the bottom ridge 32 of the end part 3 is engaged with the bottom groove 171 of the connection portion 17. As shown in FIG. 6, the canister 2 is pushed toward the needle 11 by the movement of the end part 3, and the needle 11 penetrates through the breakable portion 21 of the canister 2. The medicine 20 in the canister 2 then ejects into the patient's nose through the passage 13 of the nozzle 12.

The canister 2 can be replaced with a new one in which different medicine 20 is stored. The portable medical spray of the present invention is compact for easy carry.

The user may operate the adjustment knob 15 to drive the valve 14 to adjust the volume of the medicine 20 in the canister 2 to be ejected.

While we have shown and described the embodiment in accordance with the present invention, it should be clear to those skilled in the art that further embodiments may be made without departing from the scope of the present invention.

What is claimed is:

1. A portable medical spray comprising:
a body having a room defined therein, a needle located in the room, a nozzle formed to a top end of the body and having a passage defined through the nozzle, the passage communicating with the room and the needle, a holding portion formed to outside of the body, a connection portion extending from a bottom end of the body, a top groove and a bottom groove respectively formed to outside of the connection portion;
a canister located in the room and having medicine received therein, a breakable portion formed to a top end of the canister and located corresponding to the needle, and
an end part having a reception space defined in a top end thereof, a top ridge and a bottom ridge respectively formed to an inner periphery of the reception space, the end part movably mounted to the connection portion.

2. The portable medical spray as claimed in claim 1, wherein the passage of the nozzle of the body has a valve received therein, an adjustment knob is connected to the nozzle and operates the valve.

3. The portable medical spray as claimed in claim 1, wherein the canister is made by metal, the breakable portion of the canister is made by rubber.
